(19) United States
(12) Patent Application Publication
(10) Pub. No.: US 2011/0287437 A1
(43) Pub. Date: Nov. 24, 2011

(54) ASSAYS TO PREDICT CARDIOTOXICITY

(76) Inventors: Hans Marcus Ludwig Bitter, Montclair, NJ (US); Kyle Louis Kolaja, Montclair, NJ (US); Preeti Dhawan, San Jose, CA (US); Hirdesh Uppal, San Ramon, CA (US); Rama Rajaraja Varma, Morgan Hill, CA (US)

(21) Appl. No.: 12/713,962

(22) Filed: May 20, 2010

(51) Int. Cl. C12Q 1/68; C12Q 1/32; C12Q 1/34; C12Q 1/02

(52) U.S. Cl. 435/6.13; 435/29; 435/26; 435/23

(57) ABSTRACT

The likelihood that a compound will exhibit cardiotoxicity in vivo can be predicted using a model of in vitro assays performed on primary human cardiomyocytes.

### FIELD OF THE INVENTION

This invention relates generally to the field of toxicology. More particularly, the invention relates to methods for predicting cardiotoxicity, and methods for screening compounds for potential cardiotoxicity.

### BACKGROUND OF THE INVENTION

The heart is an adaptive organ for pumping blood, responding to changing needs by modifying contractile strength and beating rate. The cardiac myocyte is the principal cell in the heart; it coordinates contraction and has the capability to sense a large number of hormonal, neural, electrical and mechanical inputs through a variety of cell surface and nuclear receptors. Myocytes are also targets of an extraordinary number of physiological and pharmacological agents, because of the critical need to regulate contraction strength and heart rate, and their importance in several cardiovascular diseases.

Primary cells isolated from intact heart have been an important model for study because there are no cell lines that maintain the unique rod shaped morphology and complement of proteins necessary for cardiac function. In serum-free culture, adult cardiac myocytes from guinea pigs, rats, mouse and rabbits are usually quiescent and retain their viability and unique rod-shaped morphology for at least a few days. These cells maintain highly organized membrane and myofibrillar structures that support contractions induced by electrical or pharmacological stimulation, and are amenable to viral-mediated expression of exogenous proteins. But similarly successful culture of human cardiac myocytes has been more challenging and not possible, perhaps because of difficulties in enzymatic isolation of healthy myocytes and unique variables for relatively long-term culture. As a consequence, less is known about human cardiac myocyte physiology.

An understanding of cardiotoxicity and of the difficulties in predicting cardiotoxic potential requires insight into the molecular basis of the cardiac function. The understanding of molecular mechanisms of cardiotoxicity has shown that a multitude of extra cellular factors, intracellular factors, transcriptional events and signaling pathways are involved. Thus a large number of players have been shown to be key determinants in the orchestration of a multitude of these pathways to maintain normal cardiac function. Moreover, if dysregulated or inhibited, these extra cellular factors, intracellular factors, transcriptional events and signaling pathways cause the toxicities observed in adverse cardiovascular events. The development of targeted therapies, inhibitors, and drugs has shown some significant liabilities with regards to cardiotoxicity especially in the area of cancer therapy.

Recently, progress has been made in determining basic mechanisms underlying the cardiotoxicity of drugs. There are two key features to clarify for each drug, small molecule compound, ligand, or protein/biotherapuetic that show cardiotoxicity. First, determining the mechanisms of toxicity requires the identification of the specific target responsible for cardiotoxicity. The identification of targets mediating cardiotoxicity can also help to guide future drug development, because some of these molecules or proteins are likely to be ‘bystander’ targets that have no role in the disease indication that a given drug is being developed for and there is therefore no need for the drug to inhibit them. Second, there is a requirement for delineating the mechanisms of toxicity so that the signaling pathways that transduce the toxicity are identified. In some instances, the pathway that leads to cardiomyocyte dysfunction or death will not be the same as the pathway that is crucial for drug action. Therefore, strategies could be developed to block the drug-induced pathways that lead to toxicity but to leave the drug's therapeutic pathways intact.

The development of drugs that inhibit the activity of certain tyrosine kinases for cancer therapy have been associated with toxicity to the heart (Force et al., Drug Discovery Today (2008) 13(17/18), 778-784; Will et al., Toxicological Scineces (2008) 106(1), 153-161). The development of kinase inhibitors (KIs) creates many opportunities for toxicity, not only as a result of the inhibition of desired targets but, probably much more importantly, due to the inhibition of off-target kinases. Cardiotoxicity of a targeted agent was first reported for trastuzumab, the monoclonal antibody that targets the ERBB2 receptor and adverse cardiac effects have also been reported after treatment of patients with imatinib, and are mentioned in the prescribing information for dasatinib (Sprycel), sunitinib (Sutent), sorafenib (Nexavar) and bevacizumab (Avastin). Cardiotoxicity is not associated with all kinase inhibitors because it is not observed with certain other KIs, such as those that target the epidermal growth factor receptor. Therefore, cardiotoxicity needs to be determined for each agent on a case-by-case basis.

### SUMMARY OF THE INVENTION

We have now invented a method for predicting which compounds will demonstrate positive (i.e., cardiotoxicity) results in in vivo toxicity studies by testing the compounds in a set of in vitro assays on cultured primary human cardiomyocytes and determining the sets of assays that fit most accurately with the profiles of compounds that are known to exhibit cardiotoxicity in vivo.

One aspect of the invention is a method for predicting the cardiotoxicity of a compound, the method comprising providing a test compound, treating primary human cardiomyocytes with said test compound, performing at least two assays selected from the group consisting of Caspase 3/7 assay, Caspase 8 assay, Caspase 9 assay, Metabolic assay, Live Protease assay, Dead Protease assay, LDH assay, ATP assay, Lactate assay, BrdU assay, DePol assay, HyperPol assay, VO2 (STAT) assay, XTT assay, GSH assay, Lipid Perox assay, N-Lipid assay, P-Lipid assay, and ROS assay with said treated primary human cardiomyocytes, determining the results of said assays and comparing said results with results of the same assays from primary human cardiomyocytes treated with a compound known to demonstrate cardiotoxicity, wherein similar results between the test compound and the known cardiotoxic compound indicates a likelihood that the test compound will demonstrate cardiotoxicity.

Another aspect of the invention is the method for screening candidate compounds for potential cardiotoxicity, comprising providing a plurality of test compounds; treating primary human cardiomyocytes with each compound, performing at least two assays selected from the group consisting of Caspase 3/7 assay, Caspase 8 assay, Caspase 9 assay, Metabolic assay, Live Protease assay, Dead Protease assay, LDH assay, ATP assay, Lactate assay, BrdU assay, DePol assay, HyperPol assay, VO2 (STAT) assay, XTT assay, GSH assay, Lipid Perox assay, N-Lipid assay, P-Lipid assay, and ROS assay with said treated primary human cardiomyocytes, determining the results of said assays, comparing said results with results of the same assays from primary human cardiomyocytes treated with a compound known to demonstrate cardiotoxicity, wherein similar results between the test compound and the known cardiotoxic compound indicates a likelihood that the test compound will demonstrate cardiotoxicity, and rejecting compounds that demonstrate a likelihood of cardiotoxicity.

### BRIEF DESCRIPTION OF THE FIGURES

FIG. 1 Chart illustrating the assays used to generate the in vitro cardiotoxicity model and the categories assigned to the assays.

### DETAILED DESCRIPTION OF THE INVENTION

All publications cited in this disclosure are incorporated herein by reference in their entirety.

### Definitions

Unless otherwise stated, the following terms used in this Application, including the specification and claims, have the definitions given below. It must be noted that, as used in the specification and the appended claims, the singular forms “a”, “an,” and “the” include plural referents unless the context clearly dictates otherwise.

The term “cardiotoxicity” as used herein refers to compounds that cause direct or indirect injury to cardiomyocytes and the myocardium and that may manifest in certain clinical symptoms which may include: congestive heart failure, ischemia, hypotension, hypertension, arrhythmias (e.g. bradycardia), edema, QT prolongation and conduction disorders, and thromboembolism.

The term “test compound” refers to a substance which is to be tested for cardiotoxicity. The test compound can be a candidate drug or lead compound, a chemical intermediate, environmental pollutant, a mixture of compounds, and the like. The concentration of test compounds used for the assays in the present invention would vary depending upon the nature of the assay and the length of time that the test compound is exposed to the cells. In typical assays that have 24 hours or less time of exposure between the test compound and the cells, the concentration of the test compound used may range from 500 nM dose to 50 μM dose. In assays that have compound exposure time of longer than 24 hours (e.g. 48 hours or 72 hours), a lower concentration range (L) of the test compound may be used, typically from 50 nM to 5 μM. It is understood that test compound concentrations higher or lower than the concentrations disclosed herein may also be used to practice the methods of the present invention.

The term “primary human cardiomyocyte” refers to human adult cardiomyocytes derived from dissociated human heart tissue (and not from embryonic or pluripotent stem cells) which are capable of undergoing multiple passages in culture. The generation, maintenance, propagation and use of primary human cardiomyocytes are described in the concurrently filed U.S. Provisional Patent Application by Dhawan et al. entitled, “Use of Primary Human Cardiomyocytes”, U.S. Ser. No. 61/______, filed on Feb. 23, 2009 (Attorney Docket No. R0476A-PRO), which is incorporated herein by reference in its entirety.

The terms “Caspase 3/7” assay, “Caspase 8 assay” and “Caspase 9 assay” as used herein refers to assays that measure the activities of the apoptotic enzymes, caspase-3/caspase-7, caspase-8, and caspase-9, respectively. Increase in caspase activity is correlated with decrease in cell viability. Specific embodiments of the Caspase 3/7, Caspase 8 and Caspase 9 assays are described in the Examples under “Caspase 3/7 Assay”, “Caspase 8 Assay” and “Caspase 9 Assay”.

The term “Metabolic assay” as used herein refers to an assay that can measure the metabolic capacity of cells. Viable cells are expected to demonstrate high metabolic capacity whereas dead or dying cells are expected to demonstrate low metabolic capacity. A specific embodiment of the Metabolic assay is described in the Examples under “Metabolic Capacity Assay”.

The terms “Live Protease assay” and “Dead Protease assay” as used herein refer to assays that measure protease activities from live cells and dead cells, respectively. Live cells will demonstrate high live protease activity (and low or no dead protease activity) and dead cells will demonstrate high dead protease activity and low or no live protease activity). A specific embodiment of an assay that can measure both Live Protease and Dead Protease activities is described in the Examples under “Cytotoxicity-Live Cell Protease/Dead Cell Protease Assay”.

The term “LDH assay” as used herein refers to an assay that can measure the activity of the enzyme lactate dehydrogenase (LDH). Cells that are damaged tend to have leakage in the plasma membrane which results in the release of LDH and detection of extracellular LDH activity. A specific embodiment of the the LDH assay is described in the Examples under “Cytotoxicity-LDH Activity Assay”.

The term “ATP assay” as used herein refers to an assay that measures ATP levels. Live cells use ATP as energy source and are expected to have high intracellular levels of ATP while dead or dying cells have less energy needs and are expected to have low intracellular levels of ATP. A specific embodiment of the ATP assay is described in the Examples under “ATP Detection Assay”.

The term “Lactate assay” as used herein refers to an assay that measures the amount of lactate in a given environment. Cells that undergo oxidative respiration tend to have low levels of lactate whereas cells that are in stress or are in anaerobic environments will tend to have high levels of lactate. A specific embodiment of the Lactate assay is described in the Examples under “Lactate Detection Assay”.

The term “BrdU assay” as used herein refers to an assay that measures the incorporation of 5-bromo-2′-deoxy-uridine (BrdU), an analog of the nucleoside thymidine, in newly synthesized DNA. The term also encompasses any assay that measures active DNA synthesis. Since only live cells need to synthesize new DNA in order to propagate, live cells will have high levels of BrdU incorporation whereas dead or dying cells will have little or no BrdU incorporation. A specific embodiment of the BrdU assay is described in the Examples under “Cell Proliferation-DNA Synthesis Assay”.

The terms “DePol assay” and “HyperPol assay” as used herein refers to assay that measure the membrane potential of mitochondria. Mitochondrial membrane potential that is in the depolarized state signify the cells being in a toxic or damaged state and mitochondrial membrane potential that is in the hyperpolarized state signify the cells being in a stressful state. A specific embodiment of an assay that can measure the depolarization or hyperpolarization of mitochondrial membrane potential is described in the Examples under “Mitochondrial Membrane Potential Assays”.

The terms “VO2 assay” and “VO2 STAT assay” (or “VO₂ assay” and “VO₂ STAT assay”) as used herein refer to assays that measure the oxygen consumption in intact cells or isolated mitochondria. The level of oxygen consumption represents the level of mitochondrial (dys)function and toxicity as well as cell metabolism and viability. Dead or dying cells tend to have lower-than-normal oxygen consumption whereas cells under stress will have high-than-normal oxygen consumption. A specific embodiment of the VO2 (STAT) assay is described in the Examples under “Oxygen Consumption (VO₂) Assay”.

The term “XTT assay” as used herein refers to an assay that measures the activity of the succinate-tetrazolium reductase system (EC 1.3.99.1), which exists in the mitochondrial respiratory chain and is active only in viable or metabolically intact cells. A specific embodiment of the XTT assay is described in the Examples under “XTT Assay”.

The term “GSH assay” as used herein refers to an assay that measures the level of glutathione (GSH) in cells or biological samples. A change in GSH levels is important in assessment of toxicological responses and high GSH level is an indicator of oxidative stress, whereas low GSH level may indicate cell death. A specific embodiment of the GSH assay is described in the Examples under “Glutathione (GSH) Detection Assay”.

The term “Lipid Perox assay” or “Lipid Peroxidation assay” as used herein refers to an assay that measures the levels of peroxyl radicals in lipids of cells. High levels of peroxidation of lipids in cellular membranes, especially the mitochondria membrane is indicative of oxidative stress. Conversely, extremely low levels of lipid peroxidation may indicate cell damage or death. A specific embodiment of the Lipid Peroxidation assay is described in the Examples under “Lipid Peroxidation Assay”.

The terms “Neutral Lipid (N-lipid) assay” and “Phospholipid (P-Lipid) assay” as used herein refers to assays that can detect intracellular accumulation of neutral lipids and phospho lipids, respectively, generally triggered as a toxic effect of a drug. Specific embodiments of the Neutral Lipid assay and Phopholipid assay are described in the Examples under “Lipid Accumulation Assay”.

The term “ROS assay” as used herein refers to an assay that can measure the production of Reactive Oxygen Species (ROS) when live cells are placed in situations of oxidative stress. A specific embodiment of the ROS assay is described in the Examples under “Reactive Oxygen Species (ROS) Assay”.

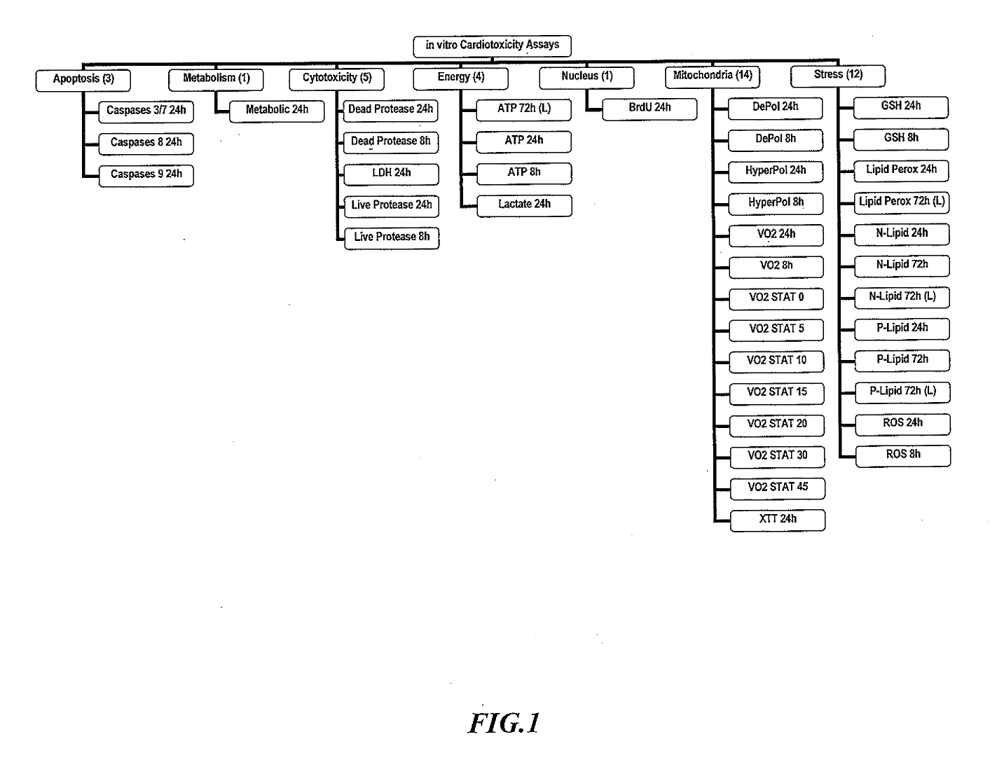

The numbers that follow the names of the assays (e.g. “ATP 8h”, “BrdU 24h” as shown in FIG. 1) refer to the length of time in hours or minutes that cells have been exposed to the test compound prior to the performance of the assay. For example, “BrdU 24h” means that the cardiomyocytes were treated with test compounds for 24 hours and then subjected to the BrdU assay, and “VO2 STAT 45” means that the cells were treated for 45 minutes with the test compounds prior to performing the Oxygen Consumption (VO2) assay.

All patents and publications identified herein are incorporated herein by reference in their entirety.

### General Method

To identify targets and signaling pathways involved in cardiotoxicity there is an urgent need to develop screening methods to provide detection of cardiotoxicity early in the drug development process. The core issue is that the current lack of high throughput procedures capable of distinguishing between drugs which are safe and those which are cardiotoxic. The main hurdle is the lack of a convenient cardiotoxicity surrogate that can easily be measured in assay formats, so the aim of the present invention was to identify assays predictive of cardiotoxicity.

The present invention provides a method for determining the likelihood that a given compound will exhibit cardiotoxicity in vivo by developing an in vitro model of cardiotoxicity. A set of compounds with known cardiotoxicity profiles were tested on cultured primary human cardiomyocytes in forty (40) in vitro assays that examined each compounds' effect on various cellular features that could be divided into seven categories: apoptosis, cytoplasmic metabolism, cytotoxicity, energy, nucleus, mitochondria and stress. All the test results for each compound in each assay were collected to generate a compendium of data that was analyzed to determine the sets of assays, either pair-wise or across all categories, that most accurately fit with a given compound's cardiotoxic profile. Several models were generated that performed with accuracies higher than 80%, with some models generating accuracies as high as 96%. Therefore, the methods of the present invention have proved to be excellent tools for the prediction of cardiotoxicity in vivo.

Candidate drugs that test positive in the methods of the present invention (i.e., that are predicted to demonstrate cardiotoxicity in vivo) would be flagged as a potential cardiotoxic compound and would be placed on hold, rejected or otherwise dropped from further development. In the case of high-throughput screening applications, such compounds can be flagged as potentially (for example, by the software managing the system in the case of an automated high-throughput system), thus enabling earlier decision making.

Thus, one can use the method of the invention to prioritize and select candidate compounds for pharmaceutical development based in part on the potential of the compound for cardiotoxicity. For example, if one has prepared a plurality of compounds (e.g., 50 or more), having similar activity against a selected target, and desires to prioritize or select a subset of said compounds for further development, one can test the entire group of compounds in the method of the invention and discard or reject all those compounds that exhibit positive signs of cardiotoxicity. This reduces the cost of pharmaceutical development, and the amount invested in any compound selected for development by identifying an important source of toxicity early on. Because the method of the invention is fast and easily automated, it enables the bulk screening of compounds that would otherwise not be possible or practical.

Environmental pollutants and the like can also be identified using the method of the invention, in which case such compounds are typically identified for further study into their toxic properties. In this application of the method of the invention, one can fractionate an environmental sample (for example, soil, water, or air, suspected of contamination) by known methods (for example chromatography), and subject said fractions to the method of the invention. Fractions that display signs of cardiotoxicity can then be further fractionated, and (using the method of the invention), the responsible toxic agents identified. Alternatively, one can perform the method of the invention using pure or purified compounds that are suspected of being environmental pollutants to determine their potential for cardiotoxicity. Because the method of the invention is fast and easily automated, it enables the bulk screening of samples that would otherwise not be possible or practical.

### EXAMPLES

### Methods

Culturing, Propagating and Plating of Primary Human Cardiomyocytes

Primary human cardiomyocytes were grown, propagated and plated for use in the in vitro assays of the present invention according to the procedures described in the concurrently filed U.S. Provisional Patent Application by Dhawan et al. entitled, “Use of Primary Human Cardiomyocytes”, U.S. Ser. No. 61/______, filed on Feb. 23, 2009 (Attorney Docket No. R0476A-PRO), which is incorporated herein by reference in its entirety.

XTT Assay

The assay was performed according to the protocol described in Cell Proliferation Kit II-XTT (Roche Applied Science, Cat. No. 11465015001). Primary human cardiomyocytes were plated on black 96-well plates and were treated with test compounds and allowed to incubate at 37° C. for 24 hours. Next, 100 μl of the Electron Coupling (EC) reagent was mixed with 5 ml of the XTT Labeling reagent and 50 μl of the EC/Labeling mixture was added to each well. The plates were gently swirled and incubated at 37° C. for 4 hours and the absorbance at 492 nm was determined by a spectrophotometer.

Oxygen Consumption (VO₂) Assay

The assay was performed in the dark according to the protocol described in the MitoXpress™ Kit (Luxcel Biosciences, Cat. No. MitoXpress-1X). Primary human cardiomyocytes on black 96-well plates were treated with test compounds for various time periods prior to the measurement of oxygen consumption: 0 minutes (VO₂ STAT 0); 5 minutes (VO₂ STAT 5); 10 minutes (VO₂ STAT 10); 15 minutes (VO₂ STAT 15); 20 minutes (VO₂ STAT 20); 30 minutes (VO₂ STAT 30); 45 minutes (VO₂ STAT 45); 8 hours (VO₂ 8 h); 24 hours (VO₂ 24 h). For the assay, 10 μl of the 1 mM MitoXpress™ probe solution was added to each well, followed immediately by the addition of 50 ml of mineral oil. The plates were incubated at 37° C. for 30 minutes and the probe's signal was measured using excitation and emission wavelengths of 381 nm and 648 nm respectively, using a fluorescence plate reader at time-resolved mode.

Reactive Oxygen Species (ROS) Assay

The assay was performed in the dark according to the protocol described under Reactive Oxygen Species (ROS) Detection Reagents (Invitrogen, Cat. No. C6827). Primary human cardiomyocytes, pre-treated with test compounds for 8 hours or 24 hours, were plated on 96-well plates and incubated with 100 μl of the ROS detection reagent, 5-(and-6)-chloromethyl-2′,7′-dichlorodihydrofluorescein diacetate, acetyl ester (CM-H₂DCFDA, 10 μM final concentration), for 30 minutes at 37° C. Production of ROS was measured by changes in fluorescence because oxidation of CM-H₂DCFDA produced the fluorescent product CM-DCF which becomes impermeable once inside the cells. The plates were read with a fluorescence plate reader at excitation and emission wavelengths of 485 nm and 535 nm respectively.

Mitochondrial Membrane Potential Assays

Following treatment with test compound for either 8 hours or 24 hours, the mitochondrial membrane potentials in the cells were measured in the dark according to the protocol described in Mitochondrial Potential Sensors (Invitrogen, Cat. No. T3168). Briefly, 100 μl of the JC-1 reagent (5,5′,6,6′-tetrachloro-1,1′,3,3′-tetraethylbenzimidazolylcarbocyanine iodide, final concentration 3.25 μM) was added to the cells in each well and the plates were incubated at 37° C. for 30 minutes. The plates were read with a fluorescence plate reader, first at red fluorescence with excitation and emission wavelengths at 535 nm and 590 nm and then at green fluorescence with excitation and emission wavelengths and 485 nm and 535 nm. Mitochondrial depolarization (DePol in FIG. 1; low membrane potential) caused by drug treatment was indicated by a decrease in the red/green fluorescence intensity ratio whereas mitochondrial hyperpolarization (HyperPol in FIG. 1; high membrane potential) from drug treatment would result in an increase in the red/green fluorescence intensity ratio.

Lipid Accumulation Assay

The intracellular accumulation of phospholipids and neutral lipids was determined by performing the assay in the dark according to the protocol described in HCS Lipid TOX™ Phospholipidosis Detection Reagents (Invitrogen, Cat. No. H34351, H34476). Primary human cardiomyocytes were plated in 96-well plates and treated simulatenously with 80 μl of the red phospholipid dye and the test compound for either 24 hours or 72 hours at 37° C. The media was then removed and 100 μl of formaldehyde fixation solution was added to each well followed by 30 minute incubation at room temperature. The fixative solution was removed and the cells were washed 2-3 times with phosphate-buffered saline, followed by the addition of 100 μl of the green neutral lipid dye. Following 30 minutes of incubation at room temperature, the plates were read in a fluorescence plate reader, first at 485 nm excitation and 535 nm emission wavelengths to detect green fluorescence (neutral lipid, N-Lipid in FIG. 1) and next at 590 nm excitation and 615 emission wavelengths to detect red fluorescence (phospholipids, P-Lipid in FIG. 1).

Lipid Peroxidation Assay

The detection of peroxyl radicals in lipids were detected following the protocols descibed in BODIPY® Lipid Probes (Invitrogen, Cat. No. D-3861). Primary human cardiomyocytes were plated in 96-well plates in the dark and treated with test compounds for 24 hours or 72 hours. After removal of media, 100 μl of BODIPY® 581/591 C₁₁ dye (4,4-difluoro-5-(4-phenyl-1,3-butadienyl)-4-bora-3a,4a-diaza-s-indacene-3-undecanoic acid, final concentration 5 μM) was added to each well and the plates were incubated at 37° C. for 30 minutes. The fluorescence at excitation wavelength of 555 nm and emission wavelength of 615 nm was measured as indication of the oxidation of the polyunsaturated portion of the dye.

Cell Proliferation-DNA Synthesis Assay

Proliferation of cardiomyocytes treated with compounds was monitored by measuring DNA synthesis as determined by BrdU (5-bromo-2′-deoxy-uridine) incorporation according to the protocol described in the Cell Proliferation ELISA, BrdU (chemiluminescence) Kit (Roche Applied Science, Cat. No. 11669915001). Briefly, primary human cardiomyocytes were plated on 96-well plates and treated with test compounds for 24 hours. The plates were then moved to a dark environment and 10 μl of BrdU labeling reagent was added in each well. Plates were incubated at room temperature for 4 hours, and after removal of media, 200 μl of FixDenat solution was added in each well, followed by 30 minutes of incubation at room temperature. The FixDenat solution was removed, and 100 μl of the Anti-BrdU-POD solution was added to each well and the plates were incubated at room temperature for 90 minutes. After removal of the solution and 2-3× washing, 100 μl of a Substrate A (luminol/4-iodophenol)/Substrate B (peroxide) mixture was added and the plates were sealed immediately and light emission was measured using a luminometer.

Cytotoxicity-LDH Activity Assay

Damage to cardiomyocytes result in plasma membrane leakage and the release of lactate dehydrogenase (LDH) into the cell culture media. Measurement of the activity of the released LDH was performed according to the protcol described in the Cytotoxicity Detection Kit^{PLUS} (LDH) (Roche Applied Science, Cat. No. 04744934001). Primary human cardiomyocytes plated on 96-well plates were treated with test compounds for 24 hours. 100 μl of the Catalyst/Dye solution (Diaphorase/NAD VINT/sodium lactate) was added to each well and the plate was incubated at room temperature for 5 minutes. 50 μl of Stop Solution was then added to each well and the plates were read for absorbance at 492 nm using a colorimetric plate reader.

Lactate Detection Assay

The assay was performed according to the protocol described in Lactate Assay Kit (BioVision, Inc. Cat. No. K607-100). Primary human cardiomyocytes plated on 96-well plates were treated with test compounds for 24 hours. Then 10 μl of culture media from each well were transferred to new 96-well plates, followed by the addition to each well of 50 μl Lactate Assay Buffer and 50 μl of Reaction Mix (Lactate Probe with Lactate Enzyme Mix). After 30 minutes incubation at room temperature, the plates were read using a fluoresence plate reader at excitation wavelength of 535 nm and emission wavelength of 590 nm.

Glutathione (GSH) Detection Assay

The assay was performed according to the protocol described in GSH-Glo™ Glutathione Assay (Promega Corporation, Cat. No. V6912). Primary human cardiomyocytes plated on 96-well plates were treated with test compounds for either 8 hours or 24 hours. The media was removed and 100 μl of 1× GSH-Glo™ Reagent (Luciferin-NT substrate and Glutathione S-Transferase mixture) was added to each well. The plates were gently shaken and incubated at room temperature for 30 minutes. Next, 100 μl of Luciferin Detection Reagent was added to the wells and the plates were incubated at room temperature for 15 minutes. Luminescence was measured using a luminescence plate reader whereby the luminescent signal is proportional to the amount of glutathione present in each well.

ATP Detection Assay

The assay was performed according to the protocol described in Cell Titer-Glo® Luminescent Cell Viability Assay (Promega Corporation, Cat. No. G7572). Primary human cardiomyocytes plated on 96-well plates were treated with test compounds for 8 hours, 24 hours or 72 hours. 100 μl of the Cell Titer-Glo® Substrate/Buffer mixture was added to each well. The plates were incubated for 10 minutes at room temperature and quantitation of ATP was measured by luminescence using a luminescence plate reader.

Cytotoxicity-Live Cell Protease/Dead Cell Protease Assay

The assay was performed according to the protocol described in MultiTox-Glo Multiplex Cytotoxicity Assay (Promega Corporation, Cat. No. G9272). Primary human cardiomyocytes plated on 96-well plates were treated with test compounds for either 8 hours or 72 hours. The live-cell protease activity was measured by addition of 50 μl of buffer containing the fluorogenic, cell-permeant peptide substrate, glycyl-phenylalanyl-amino fluorocoumarin (GF-AFC) to each well. The plates were gently shaken and incubated at 37° C. for 1 hour. Live cell fluorescence (Live Protease in FIG. 1) was measured at excitation wavelength of 405 nm and emission wavelength of 535 nm using a fluorescence plate reader. Plates were removed from the reader and dead-cell protease activity (Dead Protease in FIG. 1) was measured by adding 50 μl of buffer containing a luminogenic cell-impermeant peptide substrate, alanyl-alanyl-phenylalanyl-aminoluciferin (AAF-Glo™) to each well. Plates were incubated at room temperature for 15 minutes and the luminescent signal was measured using a luminescence plate reader.

Caspase 3/7 Assay

The assay was performed according to the protocol described in Caspase-Glo® 3/7 Assay (Promega Corporation, Cat. No. G8092). Primary human cardiomyocytes plated on 96-well plates were treated with test compounds for 24 hours and allowed to equilibrate at room temperature for 30 minutes. 100 μl of buffer containing the Caspase-Glo® 3/7 Substrate was added to each well and the plates were incubated at room temperature for 30 minutes. Plates were covered with plate sealer and luminescence was measured using a luminescence plate reader.

Caspase 8 Assay

The assay was performed according to the protocol described in Caspase-Glo® 8 Assay (Promega Corporation, Cat. No. 8202). Primary human cardiomyocytes plated on 96-well plates were treated with test compounds for 24 hours and allowed to equilibrate at room temperature for 30 minutes. Caspase-Glo® 8 lyophilized substrate and MG-132 proteasome inhibitor was mixed in buffer and 100 μl of the mixture was added to each well. The plates were covered with plate sealer and incubated for 30 minutes at room temperature. Luminescence was measured using a luminescence plate reader.

Caspase 9 Assay

The assay was performed according to the protocol described in Caspase-Glo® 9 Assay (Promega Corporation, Cat. No. 8212). Primary human cardiomyocytes plated on 96-well plates were treated with test compounds for 24 hours and allowed to equilibrate at room temperature for 30 minutes. Caspase-Glo® 9 lyophilized substrate and MG-132 proteasome inhibitor was mixed in buffer and 100 μl of the mixture was added to each well. The plates were covered with plate sealer and incubated for 30 minutes at room temperature. Luminescence was measured using a luminescence plate reader.

Metabolic Capacity Assay

The assay was performed according to the protocol described in Cell Titer-Blue® Cell Viability Assay (Promega Corporation, Cat. No. 8082). The assay uses the indicator dye resazurin to measure the metabolic capacity of viable cells which can reduce resazurin into resorufin which is highly fluorescent. Primary human cardiomyocytes plated on 96-well plates were treated with test compounds for 24 hours. 20 ml of Cell Titer-Blue Reagent was added in each well and the plates were incubated at 37° C. for 1 hour. Fluorescence was measured at excitation wavelength of 560 nm and emission wavelength of 590 nm using a fluorescence plate reader.

Analysis and Results

The aim of the analysis was to build a model using in vitro assays to predict in vivo cardiotoxicity. The analysis was carried out in several steps: first, nineteen suitable internal and marketed small molecule kinase inhibitors (SMKIs) were selected to form a training set with which to build the model; second, for each compound in the training set, a cardiotoxicity assessment (positive or negative) and data from 40 in vitro assays were acquired; and third, a statistical analysis was performed to build a predictive model.

Read-outs from 40 in vitro assays and cardiotoxicity labels were acquired for each compound in the training set (N=19). Of the nineteen compounds, ten were assessed as positive for cardiotoxicity and nine were assessed as negative as determined by information available from public literature and from internal data (Table 1). The 40 assays were assigned to one of seven categories based on function—Apoptosis, Cytoplasmic Metabolism, Cytotoxicity, Energy, Nucleus, Mitochondria, and Stress (FIG. 1).

TABLE 1

| Compounds | Cardiotoxicity | Target |
|---|---|---|
| BILN 2061 | Positive | HCV NS3 protease |
| Doxorubicin | Positive | topoisomerase II |
| Everolimus | Positive | mTOR |
| Gleevec | Positive | Abl1/2, PDGFRa/B, Kit |
| Iressa | Negative | EGFR (ERBB1) |
| Nexavar | Positive | Raf-1/B-Raf, VEGFR2/3, PDGFRa/B, |
| | | Kit, FLT3 |
| Nilotinib | Positive | Abl1/2, PDGFRa/B, Kit |
| RO-5406 | Negative | n/a |
| RO-7710 | Negative | n/a |
| RO-6145 | Negative | n/a |
| RO-3604 | Negative | h-CASEIN KINASE 1 delta-E. coli-c |
| RO-5981 | Negative | multiple |
| RO-6226 | Negative | n/a |
| Vertex | Positive | multiple |
| Sprycel | Positive | Abl1/2, PDGFRa/B, Kit, SRC family |
| Sutent | Positive | VEGFR1/2/3, Kit/PDGFRa/B, |
| | | RET, CSF-1R, FLT3 |
| Tarceva | Negative | EGFR (ERBB1) |
| Tykerb | Negative | EGFR (ERBB1), ERBB2 |
| Zactima | Positive | VEGFR/EGFR |

Pre-processing: For each assay, replicate data (n=3 to 6) were obtained at seven concentrations and the values were averaged. They were normalized against values from corresponding DMSO treated samples.

Two types of models were built: (a) Category-based Model with the goal to identify ensembles of assays, one from each category, that best predict cardiotoxicity, and (b) Overall Model with the goal was to identify pairs of assays that predict cardiotoxicity. For all analyses, cross validation was used to assess the model performance over several trials. Each trial randomly split the initial data into a training set and a test set; the training set was used to build the temporary model, and the test set was used to predict results and then verify performance. Each cross validation fold was stratified, that is, the proportion of positive to negative compounds was kept roughly equal across all folds.

For the Category-based Model, all possible combinations of category-based seven-assay panels (10,080 in all) were evaluated. For each combination, four-fold stratified cross-validations with 22 different splits of the data were performed. Prediction accuracy was calculated for each split of the data and averaged across all splits.

For the Overall Model, all possible pairs of assays without regard to category (780 combinations) were evaluated. For each combination, four-fold stratified cross-validations with 100 different splits of the data were performed. Prediction accuracy was calculated for each split of the data and averaged across all splits.

The models were ranked by average accuracy. There were 124 pairs of assays (Overall Model) with average accuracies greater than 80% (Table 2). The pair of assays in the best Overall Model were VO₂ 24 h and XTT 24 h with an average accuracy of 96%. There were 6531 category-based seven-assay panels (Category Model) with average accuracies greater than 80%. The assays in the best Category Model were Caspase 8 24 h, Metabolic 24 h, Live Protease 24 h, ATP 72 hL, VO₂ STAT 20,BrDU 24 h and ROS 24 h with an average accuracy of 92%.

TABLE 2

| Assay 1 | Assay 2 |
|---|---|
| XTT_24h | VO2_24h |
| XTT_24h | VO2_STAT_5 |
| BrdU_24h | VO2_STAT_45 |
| BrdU_24h | VO2_STAT_30 |
| XTT_24h | VO2_STAT_10 |
| XTT_24h | VO2_STAT_15 |
| XTT_24h | VO2_STAT_30 |
| Live_Protease_24h | VO2_STAT_30 |
| XTT_24h | VO2_STAT_45 |
| BrdU_24h | VO2_STAT_15 |
| Live_Protease_24h | VO2_STAT_45 |
| XTT_24h | VO2_STAT_20 |
| BrdU_24h | VO2_STAT_20 |
| Live_Protease_24h | VO2_STAT_20 |
| Live_Protease_24h | VO2_STAT_15 |
| BrdU_24h | VO2_STAT_5 |
| BrdU_24h | VO2_STAT_10 |
| Live_Protease_24h | VO2_STAT_10 |
| XTT_24h | VO2_STAT_0 |
| Live_Protease_24h | VO2_24h |
| BrdU_24h | DePol_8h |
| XTT_24h | ATP_72h_L |
| XTT_24h | N.Lipid_24h |
| XTT_24h | BrdU_24h |
| BrdU_24h | VO2_STAT_0 |
| ATP_72h_L | VO2_24h |
| Live_Protease_24h | VO2_STAT_0 |
| Live_Protease_24h | VO2_STAT_5 |
| BrdU_24h | VO2_24h |
| N.Lipid_72h | VO2_STAT_15 |
| N.Lipid_72h | VO2_STAT_20 |
| XTT_24h | DePol_8h |
| N.Lipid_72h | VO2_STAT_30 |
| BrdU_24h | Lipid_Perox_72h_L |
| BrdU_24h | P.Lipid_24h |
| BrdU_24h | N.Lipid_72h_L |
| N.Lipid_72h | VO2_STAT_45 |
| BrdU_24h | ATP_72h_L |
| BrdU_24h | N.Lipid_24h |
| ATP_8h | Caspase_8_24h |
| ATP_8h | Caspase_9_24h |
| Caspase_9_24h | Dead_Protease_8h |
| BrdU_24h | ROS_8h |
| BrdU_24h | HyperPol_8h |
| Caspase_8_24h | BrdU_24h |
| Dead_Protease_24h | VO2_STAT_15 |
| XTT_24h | Dead_Protease_24h |
| BrdU_24h | Dead_Protease_24h |
| XTT_24h | ROS_8h |
| Dead_Protease_24h | VO2_STAT_45 |
| XTT_24h | VO2_8h |
| BrdU_24h | Caspase_3/7_24h |
| BrdU_24h | Lipid_Perox_24h |
| Caspase_9_24h | VO2_STAT_30 |
| Caspase_9_24h | VO2_STAT_15 |
| Dead_Protease_24h | VO2_STAT_20 |
| BrdU_24h | Caspase_9_24h |
| BrdU_24h | VO2_8h |
| XTT_24h | P.Lipid_24h |
| Caspase_9_24h | VO2_STAT_45 |
| BrdU_24h | DePol_24h |
| N.Lipid_72h | VO2_STAT_10 |
| XTT_24h | DePol_24h |
| XTT_24h | N.Lipid_72h_L |
| Dead_Protease_24h | VO2_STAT_30 |
| BrdU_24h | Dead_Protease_8h |
| Caspase_9_24h | VO2_STAT_20 |
| BrdU_24h | ATP_8h |
| BrdU_24h | LDH_24h |
| ATP_72h_L | Live_Protease_24h |
| BrdU_24h | Live_Protease_24h |
| Caspase_9_24h | VO2_STAT_10 |
| Dead_Protease_24h | Lipid_Perox_72h_L |
| Caspase_9_24h | VO2_STAT_5 |
| P.Lipid_72h | VO2_24h |
| BrdU_24h | Metabolic_24h |
| Dead_Protease_24h | VO2_STAT_0 |
| BrdU_24h | ATP_24h |
| Dead_Protease_8h | VO2_STAT_30 |
| Dead_Protease_8h | VO2_STAT_15 |
| XTT_24h | Metabolic_24h |
| Dead_Protease_24h | VO2_STAT_10 |
| XTT_24h | ATP_8h |
| Dead_Protease_24h | VO2_STAT_5 |
| ATP_72h_L | VO2_STAT_45 |
| XTT_24h | Dead_Protease_8h |
| ATP_8h | VO2_STAT_30 |
| XTT_24h | Caspase_3/7_24h |
| BrdU_24h | N.Lipid_72h |
| Dead_Protease_8h | VO2_STAT_20 |
| BrDU_24h | P.Lipid_72h |
| XTT_24h | Caspase_8_24h |
| XTT_24h | Caspase_9_24h |
| ATP_8h | Lipid_Perox_72h_L |
| ATP_8h | VO2_STAT_20 |
| ATP_8h | VO2_STAT_15 |
| BrdU_24h | P.Lipid_72h_L |
| ATP_8h | VO2_STAT_10 |
| ATP_8h | VO2_STAT_0 |
| BrdU_24h | Lactate_24h |
| ATP_72h_L | VO2_STAT_20 |
| N.Lipid_72h | VO2_STAT_5 |
| Caspase_8_24h | VO2_STAT_30 |
| N.Lipid_72h | VO2_STAT_0 |
| Dead_Protease_8h | VO2_STAT_10 |
| XTT_24h | Lipid_Perox_24h |
| ATP_8h | VO2_STAT_45 |
| Live_Protease_24h | N.Lipid_24h |
| Dead_Protease_8h | VO2_STAT_45 |
| XTT_24h | Lipid_Perox_72h_L |
| Dead_Protease_24h | Metabolic_24h |
| XTT_24h | HyperPol_8h |
| ATP_72h_L | VO2_STAT_15 |
| ATP_8h | VO2_STAT_5 |
| Caspase_8_24h | Live_Protease_24h |
| Caspase_8_24h | VO2_STAT_15 |
| Caspase_9_24h | Live_Protease_24h |
| BrdU_24h | ROS_24h |
| Caspase_8_24h | VO2_STAT_10 |
| Caspase_8_24h | Dead_Protease_8h |
| ATP_72h_L | VO2_STAT_30 |
| Caspase_9_24h | VO2_24h |
| XTT_24h | LDH_24h |
| Caspase_8_24h | VO2_STAT_5 |

Given in vitro assay data for a compound, the models are used to predict whether that compound will be cardiotoxic. The information from the model results would be useful as a pre-screening for compounds, given the assessment difficulty and lack of mechanistic understanding of cardiotoxicity. Based on a preliminary training set of compounds with known cardiotoxicity assessment, there are several models which perform with accuracies greater than 80%. With 50% accuracy being equivalent to random classification, this model has performed well and has proved its utility in predicting cardiotoxicity.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective spirit and scope of the present invention. All such modifications are intended to be within the scope of the claims appended hereto.

What is claimed:

1. A method for predicting the cardiotoxicity of a compound, said method comprising:
a) providing a test compound;
b) treating primary human cardiomyocytes with said test compound;
c) performing at least two assays selected from the group consisting of Caspase 3/7 assay, Caspase 8 assay, Caspase 9 assay, Metabolic assay, Live Protease assay, Dead Protease assay, LDH assay, ATP assay, Lactate assay, BrdU assay, DePol assay, HyperPol assay, VO2 (STAT) assay, XTT assay, GSH assay, Lipid Perox assay, N-Lipid assay, P-Lipid assay, and ROS assay with said treated primary human cardiomyocytes;
d) determining the results of said assays; and
e) comparing said results with results of the same assays from primary human cardiomyocytes treated with a compound known to demonstrate cardiotoxicity,
wherein similar results between the test compound and the known cardiotoxic compound
indicates a likelihood that the test compound will demonstrate cardiotoxicity.

2. The method of claim 1, wherein one of said at least two assays in step c) is the BrdU assay and the other of said at least two assays is selected from the group consisting of Caspase 3/7 assay, Caspase 8 assay, Caspase 9 assay, Metabolic assay, Live Protease assay, Dead Protease assay, LDH assay, ATP assay, Lactate assay, DePol assay, HyperPol assay, VO2 (STAT) assay, XTT assay, Lipid Perox assay, N-Lipid assay, P-Lipid assay, and ROS assay.

3. The method of claim 1, wherein one of said at least two assays in step c) is the XTT assay and the other of said at least two assays is selected from the group consisting of Caspase 3/7 assay, Caspase 8 assay, Caspase 9 assay, Metabolic assay, Dead Protease assay, LDH assay, ATP assay, BrdU assay, DePol assay, HyperPol assay, VO2 (STAT) assay, Lipid Perox assay, N-Lipid assay, P-Lipid assay and ROS assay.

4. The method of claim 1, wherein one of said at least two assays in step c) is the VO2 (STAT) assay and the other of said at least two assays is selected from the group consisting of Caspase 8 assay, Caspase 9 assay, Live Protease assay, Dead Protease assay, ATP assay, BrdU assay, XTT assay, N-Lipid assay, P-Lipid assay, and ROS assay.

5. The method of claim 1, wherein said at least two assays in step c) are selected from the pair of assays shown on Table 2.

6. The method of claim 1, wherein said at least two assays in step c) are selected from the group consisting of XTT assay and VO2_{—}24 h assay, and BrdU assay and VO2_STAT_{—}45 assay.

7. A method for screening compounds for potential cardiotoxicity, said method comprising:
a) providing a plurality of test compounds;
b) treating primary human cardiomyocytes with each test compound;
c) performing at least two assays selected from the group consisting of Caspase 3/7 assay, Caspase 8 assay, Caspase 9 assay, Metabolic assay, Live Protease assay, Dead Protease assay, LDH assay, ATP assay, Lactate assay, BrdU assay, DePol assay, HyperPol assay, VO2 (STAT) assay, XTT assay, GSH assay, Lipid Perox assay, N-Lipid assay, P-Lipid assay, and ROS assay with said treated primary human cardiomyocytes;
d) determining the results of said assays;
e) comparing said results with results of the same assays from primary human cardiomyocytes treated with a compound known to demonstrate cardiotoxicity, wherein similar results between the test compound and the known cardiotoxic compound indicates a likelihood that the test compound will demonstrate cardiotoxicity.
f) rejecting compounds that demonstrate a likelihood of cardiotoxicity.

8. The method of claim 7, wherein one of said at least two assays in step c) is the BrdU assay and the other of said at least two assays is selected from the group consisting of Caspase 3/7 assay, Caspase 8 assay, Caspase 9 assay, Metabolic assay, Live Protease assay, Dead Protease assay, LDH assay, ATP assay, Lactate assay, DePol assay, HyperPol assay, VO2 (STAT) assay, XTT assay, Lipid Perox assay, N-Lipid assay, P-Lipid assay, and ROS assay.

9. The method of claim 7, wherein one of said at least two assays in step c) is the XTT assay and the other of said at least two assays is selected from the group consisting of Caspase 3/7 assay, Caspase 8 assay, Caspase 9 assay, Metabolic assay, Dead Protease assay, LDH assay, ATP assay, BrdU assay, DePol assay, HyperPol assay, VO2 (STAT) assay, Lipid Perox assay, N-Lipid assay, P-Lipid assay and ROS assay.

10. The method of claim 7, wherein one of said at least two assays in step c) is the VO2 (STAT) assay and the other of said at least two assays is selected from the group consisting of Caspase 8 assay, Caspase 9 assay, Live Protease assay, Dead Protease assay, ATP assay, BrdU assay, XTT assay, N-Lipid assay, P-Lipid assay, and ROS assay.

11. The method of claim 7, wherein said at least two assays in step c) are selected from the pair of assays shown on Table 2.

12. The method of claim 1, wherein said at least two assays in step c) are selected from the group consisting of XTT assay and VO2_{—}24 h assay, and BrdU assay and VO2_STAT_{—}45 assay.
